SEQUENCE LISTING (5 sequences)

SEQ ID NO: 1 - protein, 577 aa, Brachypodium distachyon
MAMSTRCHAPLAACVFLVCAVSMAVYTAFPLSMSPCTNSLSRALLAISGLDPYITSCADHDDASAARLSDGGGSDNIIGGPIVTNLLTCGNATLPPHALPPFYCCPPMTTAEPINFTFPDPSEPLRVRRPAHAVGAEYMAKYERVIALMKALPHSDPRSFYQVANIHCAYCTTSYRQANPKLGVQIHFSWLFFTFHRAHLYFFERIAAKLLGEPEFALPFWSWDVPEGMRMPVEFANSSSVLYDPIRNPSHAPPKLVDLDFLGPEKNFTDEQQIQHNLRVMYKQMIGNAALPSLFHGQPYRAGQNDMPGAGTVELAPHNTVHTWTGDITLPNVENMGDYYSAGRDPIFYPHHNNIDRLWQAWRDAGVARGYRGHVDFTDPDWLDSSFLFYNEDARLVRITVRDVLDTEKLRYTHAGVGMPWLDAKPPTTPNVNTKKGSLKSVRFPVSLDAAVSAEVRRPRVLRSRHEKMAQEEVLVVEGVETNGNELVKFDVFVNAMEHEKVEAGGRELAGTFVSMKQPSMDHRTGKRKPMKTSMRVALNELLEDLGADGDESVTVTLVPRRGNVRIGGLRIVYMTE

SEQ ID NO: 2 - protein, 592 aa, Brachypodium distachyon
MASLSQLIARPTTTVQCYPWSPCSNSSSLKPRRAAGRVRCTLSTDATGGRAEHDGPRLDRRDVLLRLGTLGASATAGLLSSPRLAGAAPVATPDISSCGKPDLGLPPNANLLTCCPPPSNALPVDFSPPDASTPLRTRPAAHSVGADYVAKLNRAMAAMKALPAEDPRSFAAQASIHCAYCNGSYGVEGFPGSDLQVHNSWLFLPFHRCYLYFFERILGSLIGDPSFAMPFWNWDAPGGMRMPAMYVDPKSPLFDPRRDARHAPPELINLDYNGREPTFTDRQQVDHNLRVMYRQMVSLSPTPSLFFGGAYRAGDEPDQGPGPMENIPHGPVHIWCGDPNQPAGEDMGNFYSAGRDPLFYAHHGNIDRMWSVWKGLDPRRHRDLTDPDWLDSSFLFYDETPKLVRIRVRDVLDTDRLRYRFQDVPMPWTTARPTVTPRARSFGTPTAVAASAKKATKFPIMLDSATSVTVRRPVSSKRSKLEKSAKEEVLVIGGIEVDMDIAAKFDVFVNAGDDHAAVGSGGRELAGSFVSVPHRHRHDKKEKKIKTKLRLALNEQLEDLDAEGDESVVVTLVPRQGKGKVKIGSVKIELID

SEQ ID NO: 3 - protein, 583 aa, Brachypodium distachyon
MKAEAMASSRCGPLGACVLLICAVATAVYTAFPVSVNPCTYSLPRALLAVSGLDPYIVSCAADEDAFTAPLSNGGNDDKNIGGPIVTNLLTCGKPKLPPHALPPFYCCPPMSASEPIDFTFPDPSEPLRVRRPAHAVGAEYMAKYERAIALMKALPDSDPRSFYQMANIHCAYCTGSYRQTAHRELNVQIHFSWFFFAFHRAYLYFFERIAAKLLGEPGFAVPFWSWDVPEGMRMPVEFANASSPLYDPVRNPRHAPPKVVDLEFVRSSVDDKFTDEQQIQQNLRVMYKQMISNAALPSLFHGQPYRAGESDRPGAGTVELFPHNTMHTWTGDLARPSVENMGVYYSAGRDPIFYPHHNNIDRLWEVWRDVGAARGYRGHVDFTDPDWLDSSFLFYDEEARLVRITVRDVLDIDKLRYAYDGVGTPWLDAKPPATPNVNTKKGLLKSVRFPVSLDDVAVTAEVRRPRVLRSRREKEVQEEVLVIDGIETDGADMVKFDVYVNAVEYEKVEPGGREMAGSFVCLMHPSMDGTGKGMGIQTSMRVALNELLEDLGADGDDSVTVTLVPRNGKVSIGGLRIVYMME

SEQ ID NO: 4 - protein, 533 aa, Brachypodium distachyon
MAPTSMCFDAINGAAAAQNGTAPVLATKPAAQALQCPNALNADDFRRQGHQVIDFIAEYYGGMADYPVHPSVTPGFLRNLLPASAPSRAEPDAFSSALKDIRDHILPGMTHWQSPRHFAHFPASSSTVGALGEALTAGINVVPFTWAASPAATELEMVVVDWLGKALHLPETLLFAGGGGGTLLGTSCEAILCALVAARDRKLAEIGGRRIGDLVVYCSDQTHFAFRKAARIAGILREHIREIQTCHANMFALSATALEAAMQADVEAGLVPLFVCATVGTTQTTAVDPIGELCTVTAPHGVWVHVDAAYAGSALVCPEFRHVINGVESVDSFSMNAHKWLLTNNDCCAMWVKKPSELIAALGTEQEYILKDSASEGHDIVDYKDWTMTLTRRFRALKMWLVLRCYGIDGLREHIRSHVRMAEAFENLVRADERFEVVTDRQFALVCFRLRSPEKYGGEKTANELNRSLLEEVNAVTLGPYMSSANVGGMYMLRCAVGSTLTEDCHVTDGWKVVQDRATSILRKMEIIYSVLG

SEQ ID NO: 5 - protein, 521 aa, Brachypodium distachyon
MAPPSHFSNVAATVPVVVDKPQQCSNALDADDFRRQGHQVIDFIAEYYGGMADYPVHPSVTPGFLRNLLPASAPSRAEPDAFSSALKDIRDHILPGMTHWQSPRHFAHFPASSSTVGALGEALTAGINVVPFTWAASPAATELEMVVVDWLGKALHLPETLLFAGGGGGTLLGTSCEAILCALVAARDRKLAEIGGRRIGDLVVYCSDQTHFAFRKAARIAGILREHIREIQTCHANMFALSATALEAAMQADVEAGLVPLFVCATVGTTQTTAVDPIGELCTVTAPHGVWVHVDAAYAGSALVCPEFRHVINGVESVDSFSMNAHKWLLTNNDCCAMWVKKPSELIAALGTEQEYILKDSASEGHDIVDYKDWTMTLTRRFRALKMWLVLRCYGIDGLREHIRSHVRMAEAFENLVRADERFEVVTDRQFALVCFRLRSPEKYGGEKTANELNRSLLEEVNAVTLGPYMSSANVGGMYMLRCAVGSTLTEDCHVTDGWKVVQDRATSILRKMEIIYSVLG

(19) United States
(12) Patent Application Publication
(10) Pub. No.: US 2025/0283125 A1
(43) Pub. Date: Sep. 11, 2025

(54) PRODUCTION OF DOPAMINE IN A PLANT OR PLANT CELL

(71) Applicant: THE REGENTS OF THE UNIVERSITY OF CALIFORNIA, Oakland, CA (US)

(72) Inventors: Yezhang DING, Berkeley, CA (US); Trent R. NORTHEN, Walnut Creek, CA (US); John P. VOGEL, Walnut Creek, CA (US)

(21) Appl. No.: 19/019,208

(22) Filed: Jan. 13, 2025

Related U.S. Application Data
(60) Provisional application No. 63/620,067, filed on Jan. 11, 2024.
(60) Provisional application No. 63/744,089, filed on Jan. 10, 2025.

(51) Int. Cl. C12P 13/00; C12N 5/04; C12N 9/02; C12N 9/88; C12N 15/82

(52) U.S. Cl. CPC C12P 13/001; C12N 5/04; C12N 9/0004; C12N 9/88; C12N 15/8201; C12N 15/8243; C12Y 401/01025; C12N 2510/02

(57) ABSTRACT

The present invention provides for a genetically modified plant or plant cell capable of producing dopamine or dopamine-derived compound, comprising one or more heterologous tyrosine decarboxylases (TyDC) or one or more heterologous polyphenol oxidases (PPO).

### CROSS REFERENCE TO RELATED APPLICATIONS

This application claims priority to U.S. Provisional Patent Application Ser. Nos. 63/620,067, filed Jan. 11, 2024, and 63/744,089, filed Jan. 10, 2025, both of which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### REFERENCE TO SEQUENCE LISTING

The instant application contains a Sequence Listing which has been submitted electronically in XML format and is hereby incorporated by reference in its entirety. Said XML copy, created on Apr. 29, 2025, is named “2023-066-02 Sequence Listing.xml” and is 8,186 bytes in size.

### FIELD OF THE INVENTION

The present invention is in the field of production of dopamine in a plant.

### BACKGROUND OF THE INVENTION

Dopamine is an important neurotransmitter in the brain. In humans, it plays a wide range of functions in processes involved in motor control, motivation, reward, addition, and hormonal secretion (Olguín et al., 2016). The dysregulation of the dopaminergic system has been implicated in human diseases including Parkinson's disease, depression, and attention deficit hyperactivity disorder (Olguín et al., 2016). Dopamine serves as a human drug for several important diseases, such as Parkinson's disease. Dopamine and its derivatives are prescription medicines used to treat the symptoms of low blood pressure, and low cardiac output and improves blood flow to the kidneys as well as many others. Many plant species also produce dopamine and its related compounds. Existing evidence indicates that dopamine plays a significant role in plant growth and stress responses (FIG. 1; Liu et al., 2020). Dopamine is also a precursor in the biosynthesis of important/valuable molecules such as morphine (Galanie et., 2015). Unfortunately, despite all of these benefits and commercial interest, the dopamine biosynthetic pathway in plants has remained unclear, thus limiting our complete understanding of biological functions of this pathway in plants as well as prohibiting bioengineering this pathway in plants for human and animal health.

### SUMMARY OF THE INVENTION

The present invention provides for a genetically modified plant or plant cell capable of producing dopamine or dopamine-derived compound, comprising one or more heterologous tyrosine decarboxylases (TyDC) or one or more heterologous polyphenol oxidases (PPO). In some embodiments, the genetically modified plant or plant cell has an improved plant stress resilience.

The present invention provides for a method for constructing a genetically modified plant or plant cell of the present invention, comprising: (a) introducing one or more nucleic acids or polynucleotides comprising the open reading frame(s) (ORF) encoding one or more heterologous tyrosine decarboxylases (TyDC) or one or more heterologous polyphenol oxidases (PPO), wherein each ORF is operatively linked to a promoter capable of expressing the TyDC(s) and/or PPO(s) into the plant or plant cell; and (b) the one or more nucleic acids or polynucleotides stably reside in the plant or plant cell. In some embodiments, the one or more nucleic acids or polynucleotides stably reside in the plant or plant cell either in a vector or integrated into a plant genome of the plant or plant cell. In some embodiments, each promoter is independently a constitutive promoter, inducible promoter, or cell type-specific promoter.

In some embodiments, the nucleic acid or polynucleotide is part of a vector. In some embodiments, the vector is introduced into the plant or plant cell, and the vector is capable of stably integrating into a chromosome or genome of the plant or plant cell or stably residing in the plant or plant cell. In some embodiments, the vector is an expression vector.

The present invention provides for a method for producing dopamine or dopamine-derived compound, comprising: (a) providing a genetically modified plant or plant cell of the present invention; (b) culturing or growing the genetically modified plant or plant cell thereby the genetically modified plant or plant cell produces dopamine or dopamine-derived compound; and, (c) optionally separating or purifying the dopamine or dopamine-derived compound from the rest of the genetically modified plant or plant cell.

In some embodiments, the method further comprises first constructing the genetically modified plant or plant cell of the present invention using the method provided herein. In some embodiments, the method further comprises administering the separated or purified dopamine or dopamine-derived compound to a subject of such treatment.

In some embodiments, the method further comprises converting the separated or purified dopamine into a dopamine-derived compound, or the separated or purified dopamine-derived compound into a second dopamine-derived compound. In some embodiments, the dopamine-derived compound, or second dopamine-derived compound, is morphine and/or codeine, or a polydopamine, or any other dopamine-derived compound shown in FIG. 11.

In some embodiments, the method further comprises converting the separated or purified dopamine or dopamine-derived compound into a biomaterial.

In some embodiments, the plant or plant cell has less melanin produced thereby resulting a reduced brown color.

In some embodiments, the plant or plant cell are modified thereby enhancing the plant or plant cell's capability to store carbon in soil, and optionally to store nitrogen in soil.

In some embodiments, the plant or plant cell are modified thereby reducing or removing phenols and other organic compounds from wastes.

In some embodiments, the invention comprises: (1) Engineering/breeding plants with altered TyDC and PPO to produce dopamine to improve plant stress resilience. (2) Engineering/breeding plants with altered TyDC and PPO to produce dopamine for human and animal health applications. (3) Engineering/breeding plants with altered TyDC and PPO to produce dopamine to selectively enhance the growth of beneficial microbes that use dopamine. (4) Engineering/breeding plants with altered TyDC and PPO for enhanced soil carbon storage as well as potential soil nitrogen storage. (5) Engineering/breeding plants with altered TyDC and PPO to produce polydopamine for use in the construction of biomaterials. (6) Engineering/breeding plants with altered TyDC and PPO to reduce brown color. (7) Engineering microorganisms with TyDC and PPO to produce dopamine as precursors for synthesizing valuable dopamine-derived compounds, such as morphine and codeine. (8) Engineering microorganisms with TyDC and PPO to produce dopamine for use in the construction of biomaterials. (9) Engineering microorganisms with TyDC and PPOs for enhancing soil carbon storage as well as potential soil nitrogen storage. (10) Engineering microorganisms with TyDC and PPO for removing phenols and other organic compounds from wastes.

In some embodiments, chemical inducers or senescence-related promoters can be used to control expression of dopamine pathway enzymes (such as TyDC and/or PPO). In some embodiments, methyltransferases can be used to protect plants with senescence-related promoters from leakage (toxicity), wherein the plants to turn themselves into polydopamine when they senesce (but not before senesce).

Brachypodium distachyon plants can produce extremely high levels of dopamine (>5 mg/g Fresh Weight) under certain growth conditions. Multi-omics data analysis in combination with metabolite-based genome wide association studies enabled us to identify several candidate enzymes for dopamine biosynthesis, including two tyrosine decarboxylases (BdTyDC2 & 3) and three polyphenol oxidases (BdPPO1, BdPPO2, and BdPPO3). Interestingly, these enzyme-encoding genes form a cluster on chromosome 2 in Brachypodium distachyon. Transcriptomic data analysis demonstrated that these genes except BdPPO3 are predominantly expressed in roots. Subsequent in vivo enzymatic assays confirm that combination of BdTyDC2 or BdTyDC3 with BdPPO1 or BdPPO2 resulted in dopamine production, proving that these enzymes are involved in plant dopamine biosynthesis.

Dopamine can undergo oxidative self-polymerization in vitro to yield polydopamine, which is a robust universal coating material with numerous promising properties, such as antimicrobial activity (FIG. 2; Ryu et al., 2018). The polymerization process can be expedited by enzymes, like laccases (Li et al., 2018), and can involve other phenols and peptides (de Nobili et al., 2020). Consequently, melanins derived from dopamine with the incorporation of phenols and/or peptides emerge as highly promising targets for enhancing soil carbon storage, and/or nitrogen storage. Example 1 herein also shows the production of polydopamine in planta. It is found that transient overexpression of a tyrosine decarboxylase (BdTyDC2 or BdTyDC3) together with a polyphenol oxidase (BdPPO1, BdPPO2, or BdPPO3) in Nicotiana benthamiana yielded the production of extremely high levels of dopamine (FIG. 6; >3 mg/g Fresh Weight), and the production of polydopamine and/or or dopamine containing biopolymers. It was also found that damaging the tobacco leaves transiently expressing the dopamine pathway could lead to the production of insoluble melanins (FIG. 8).

In some embodiments, the plants or plant cells can produce equal to or more than 3 mg/g Fresh Weight, equal to or more than 4 mg/g Fresh Weight, equal to or more than 5 mg/g Fresh Weight, or equal to or more than 6 mg/g Fresh Weight,

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Potential roles of dopamine in plants.

FIG. 2, Potential applications of polydopamine in industry, environment, and human health.

FIG. 3. Production of dopamine in Brachypodium roots. a, Representative LC/MS total ion chromatograms (TICs) of Brachypodium root extracts and a dopamine standard. b, Average quantity (n=3) of dopamine in Brachypodium fresh roots, which was estimated based on an external dopamine standard curve.

FIG. 4. Discovery of candidate dopamine biosynthetic genes in Brachypodium distachyon. a, Manhattan plot of a Brachypodium GWAS using the ratio of dopamine to tyramine in roots as a mapping trait. Negative log 10-transformed P values from the general linear model are plotted on the y axis. The dashed line denotes the 5% Bonferroni-corrected threshold, with the most statistically significant SNPs located on chromosome 2. b, A regional Manhattan plot representing a ‘zoomed-in’ view of the signal between 51.05 Mb and 51.15 Mb on Chr.2 (Bd21-3 v1.1), and each dot representing a single SNP. c, Genes located inside the region with SNPs with Negative log 10-transformed P values larger than the threshold. Among them, BdiBd21-3.2G0666400 encodes a putative polyphenol oxidase, BdPPO1, with a predicted function in converting tyramine to dopamine.

FIG. 5. Expression of the BdTyDC and BdPPO family genes in roots and shoots of Brachypodium seedlings. Transcript abundance of the BdTyDC family genes TyDC1 to TyDC4 (a) and the BdPPO family genes BdPPO1 to BdPPO6 (b) derived from RNA-seq analyses of Brachypodium roots and shoots harvested from hydroponically grown Bd21-3 at week 2 and week 4. Gene expression is given as FPKM (Fragments per kilo base of transcript per million mapped fragments). c, Two BdTyDC genes (BdTyDC2 & BdTyDC3) and three BdPPO genes (BdPPO1, BdPPO2, & BdPPO3) form a cluster on Brachypodium chromosome 2 (Bd21-3 v1.1). Nomenclature: BdTyDC1: BdiBd21-3.2G0028800, TyDC2: BdiBd21-3.2G0653800, BdTyDC3: BdiBd21-3.2G0654700, BdTyDC4: BdiBd21-3.5G0285466. BdPPO1: BdiBd21-3.2G0666400, BdPPO2: BdiBd21-3.2G0667800, BdPPO3: BdiBd21-3.2G0668300, BdPPO4: BdiBd21-3.3G0199700, BdPPO5: BdiBd21-3.3G0200300, BdPPO6: BdiBd21-3.5G0291800.

FIG. 6. Characterization of Brachypodium TyDC and PPO family genes. a, Representative LC/MS extraction ion chromatograms (XIC) of tyramine (m/z, 138.091) in N. benthamiana leaves transiently expressing Brachypodium TyDC genes as labelled in the figure. b, LC/MS peak height of dopamine (m/z, 154.086) extracted from N. benthamiana leaves coexpressing Brachypodium PPOs (PPO1-PPO6) individually with BdTyDC3 for 3 days.

FIG. 7. BdPPO1 homologs catalyze dopamine formation in plants. Average quantity (n=3) of dopamine in N. benthamiana, which was estimated based on an external dopamine standard curve. BdPPO1 and its homologs from Zea mays (ZmPPO, NP_001147867) and Papaver somniferum (PsPPO1: XP_026418439; PsPPO2: XP_026449197, PsPPO3: XP 026453118; and PsPPO4: XP_026425793) were coexpressed with BdTyDC2 using transient, Agrobacterium-mediated expression in N. benthamiana. ZmPPO, PsPPO1, and PsPPO4 show the enzymatic activity for dopamine biosynthesis. The genes ZmPPO, PsPPO1, PsPPO2, PsPPO3, and PsPPO4 were synthesized with codon-optimized sequences.

FIG. 8. Formation of dopamine polymers. N. benthamiana leaves transiently expressing either BdPPO or BdTyDC or both, were extracted using 70% methanol. The extracts were centrifuged, and the resulting supernatants were left at room temperature for 2 weeks (a). The black precipitate was not soluble in both 70% methanol (b) and water (c).

FIG. 9. Characterization of the Brachypodium ppo1 mutant. a, The color of root extracts. Roots of Hydroponically grown Brachypodium plants, Bd21-3, the wild-type sibling, and ppo1, were grounded and extracted with 70% methanol. b and c show root biomass (b) and shoot biomass (c) of four-week-old hydroponically grown Brachypodium plants. d and e show the relative levels of dopamine detected by LC/MS in plant roots (d) and shoots (e) of Bd21-3, the wild-type sibling, and ppo1.

FIG. 10. Identification of methyltransferases in the prevention of dopamine polymer formation. a, The colors of 0.1 mM dopamine and 3-methoxydopamine solutions at room temperature for 3 days; The black color indicates the formation of dopamine polymers, such as polydopamine; b, Manhattan plot of a Brachypodium GWAS using the level of 3-methoxytyramine in roots as a mapping trait. Negative log 10-transformed P values from the general linear model are plotted on the y axis. The dashed line denotes the 5% Bonferroni-corrected threshold, with the most statistically significant SNPs located on chromosome 3. c, A regional Manhattan plot representing a ‘zoomed-in’ view of the signal between 41.55 Mb and 41.70 Mb on Chr.3 (Bd21-3 v1.1), and each dot representing a single SNP. d, Expression of five methyltransferase-encoding genes located inside the region, derived from RNA-seq analyses of Brachypodium roots and shoots harvested from hydroponically grown Bd21-3 at week 2 and week 4.

FIG. 11. Examples of dopamine derived compounds, including polydopamine.

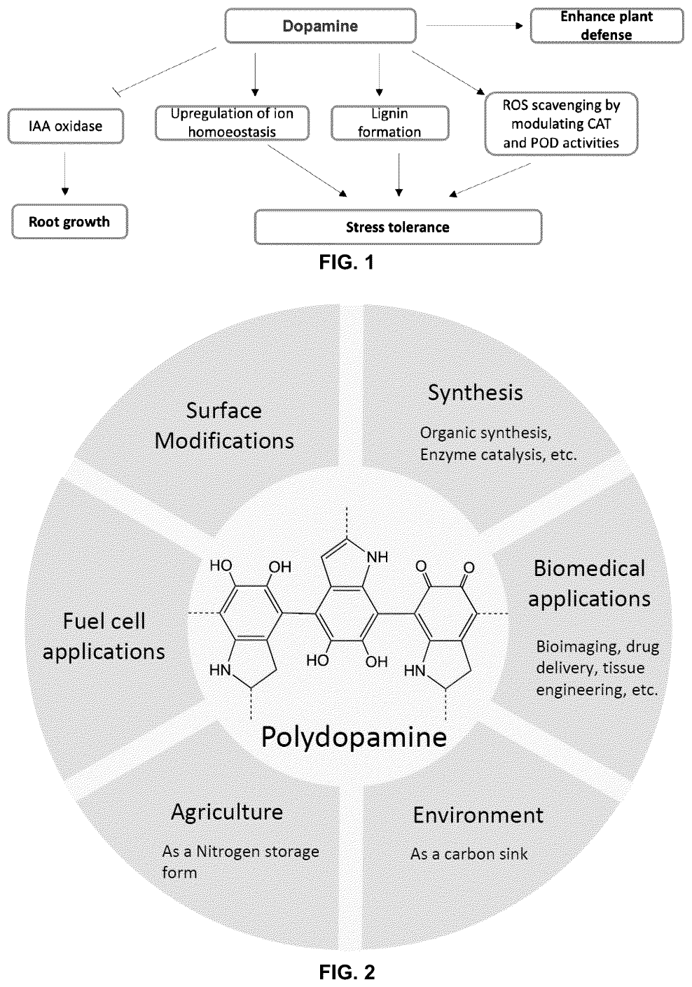

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A “constitutive promoter” is one that is capable of initiating transcription in nearly all cell types, whereas a “cell type-specific promoter” initiates transcription only in one or a few particular cell types or groups of cells forming a tissue. In some embodiments, the promoter is secondary cell wall-specific and/or fiber cell-specific. A “fiber cell-specific promoter” refers to a promoter that initiates substantially higher levels of transcription in fiber cells as compared to other non-fiber cells of the plant. A “secondary cell wall-specific promoter” refers to a promoter that initiates substantially higher levels of transcription in cell types that have secondary cell walls, e.g., lignified tissues such as vessels and fibers, which may be found in wood and bark cells of a tree, as well as other parts of plants such as the leaf stalk. In some embodiments, a promoter is fiber cell-specific or secondary cell wall-specific if the transcription levels initiated by the promoter in fiber cells or secondary cell walls, respectively, are at least 3-fold, 4-fold, 5-fold, 6-fold, 7-fold, 8-fold, 9-fold, 10-fold, 50-fold, 100-fold, 500-fold, 000-fold higher or more as compared to the transcription levels initiated by the promoter in other tissues, resulting in the encoded protein substantially localized in plant cells that possess fiber cells or secondary cell wall, e.g., the stem of a plant. Non-limiting examples of fiber cell and/or secondary cell wall specific promoters include the promoters directing expression of the genes IRX1, IRX3, IRX5, IRX7, IRX8, IRX9, IRX10, IRX14, NST1, NST2, NST3, MYB46, MYB58, MYB63, MYB83, MYB85, MYB103, PAL1, PAL2, C3H, CcOAMT, CCR1, F5H, LAC4, LAC17, CADc, and CADd. See, e.g., Turner et al 1997; Meyer et al 1998; Jones et al 2001; Franke et al 2002; Ha et al 2002; Rohde et al 2004; Chen et al 2005; Stobout et al 2005; Brown et al 2005; Mitsuda et al 2005; Zhong et al 2006; Mitsuda et al 2007; Zhong et al 2007a, 2007b; Zhou et al 2009; Brown et al 2009; McCarthy et al 2009; Ko et al 2009; Wu et al 2010; Berthet et al 2011. In some embodiments, a promoter is substantially identical to a promoter from the lignin biosynthesis pathway. A promoter originated from one plant species may be used to direct gene expression in another plant species.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

In some embodiments, one or more of the enzymes, including TyDC and/or PPO is an engineered enzyme, or homologous, mutant or variant enzymes having or comprising the same enzymatic activity as the corresponding wild-type enzyme, with an amino acid sequence having equal to or more than 70%, 75%, 80%, 85%, 90%, 95%, or 99% identity to the amino acid sequence of the corresponding wild-type enzyme, such as the specific enzymes described herein (including SEQ ID NO:1 to SEQ ID NO:5.

In some embodiments, the TyDC is any wild-type TyDC of any species, such as any microbial species, such as any yeast or bacterial species. In some embodiments, the TyDC has an enzymatic activity identical to a Brachypodium distachyon TyDC. In some embodiments, the tyrosine decarboxylase (TyDC) is a Brachypodium distachyon TyDC (BdTyDC). In some embodiments, the BdTyDC is BdTyDC2 or BdTyDC3.

In some embodiments, the PPO is any wild-type PPO of any species, such as any microbial species, such as any yeast or bacterial species. In some embodiments, the PPO has an enzymatic activity identical to a Brachypodium distachyon PPO. In some embodiments, the polyphenol oxidase (PPO) is a Brachypodium distachyon PPO (BdPPO). In some embodiments, the BdPPO is BdPPO1, BdPPO2, or BdPPO3.

The amino acid sequence of BdPPO1 (BdiBd21-3.2G0666400_PPO1) is as follows:

```
  (SEQ ID NO: 1)
MAMSTRCHAPLAACVFLVCAVSMAVYTAFPLSMSPCTNSLSRALLAISGLDPYITSCADHDDASAARLSDGGGSDNIIGGP
IVTNLLTCGNATLPPHALPPFYCCPPMTTAEPINFTFPDPSEPLRVRRPAHAVGAEYMAKYERVIALMKALPHSDPRSFYQ
VANIHCAYCTTSYRQANPKLGVQIHFSWLFFTFHRAHLYFFERIAAKLLGEPEFALPFWSWDVPEGMRMPVEFANSSSVLY
        * * *
DPIRNPSHAPPKLVDLDFLGPEKNFTDEQQIQHNLRVMYKQMIGNAALPSLFHGQPYRAGQNDMPGAGTVELAPHNTVHTW
                                                                                                                                                    * *
TGDITLPNVENMGDYYSAGRDPIFYPHHNNIDRLWQAWRDAGVARGYRGHVDFTDPDWLDSSFLFYNEDARLVRITVRDVL
                            * *
DTEKLRYTHAGVGMPWLDAKPPTTPNVNTKKGSLKSVRFPVSLDAAVSAEVRRPRVLRSRHEKMAQEEVLVVEGVETNGNE
LVKFDVFVNAMEHEKVEAGGRELAGTFVSMKQPSMDHRTGKRKPMKTSMRVALNELLEDLGADGDESVTVTLVPRRGNVRI
GGLRIVYMTE
Note: Cu cation A: H167, H187 and H196; Cu cation B: H318, H322 and H352.
```

The amino acid sequence of BdPPO2 (BdiBd21-3.2G0667800_PPO2) is as follows:

```
(SEQ ID NO: 2)
MASLSQLIARPTTTVQCYPWSPCSNSSSLKPRRAAGRVRCTLSTDATGGRAEHDGPRLDRRDVLLRLGTLGASATAGLLSS
PRLAGAAPVATPDISSCGKPDLGLPPNANLLTCCPPPSNALPVDFSPPDASTPLRTRPAAHSVGADYVAKLNRAMAAMKAL
PAEDPRSFAAQASIHCAYCNGSYGVEGFPGSDLQVHNSWLFLPFHRCYLYFFERILGSLIGDPSFAMPFWNWDAPGGMRMP
                            * * *
AMYVDPKSPLFDPRRDARHAPPELINLDYNGREPTFTDRQQVDHNLRVMYRQMVSLSPTPSLFFGGAYRAGDEPDQGPGPM
ENIPHGPVHIWCGDPNQPAGEDMGNFYSAGRDPLFYAHHGNIDRMWSVWKGLDPRRHRDLTDPDWLDSSFLFYDETPKLVR
        * * * *
IRVRDVLDTDRLRYRFQDVPMPWTTARPTVTPRARSFGTPTAVAASAKKATKFPIMLDSATSVTVRRPVSSKRSKLEKSAK
EEVLVIGGIEVDMDIAAKFDVFVNAGDDHAAVGSGGRELAGSFVSVPHRHRHDKKEKKIKTKLRLALNEQLEDLDAEGDES
VVVTLVPRQGKGKVKIGSVKIELID
```

The amino acid sequence of BdPPO3 (BdiBd21-3.2G0668300_PPO3) is as follows:

```
(SEQ ID NO: 3)
MKAEAMASSRCGPLGACVLLICAVATAVYTAFPVSVNPCTYSLPRALLAVSGLDPYIVSCAADEDAFTAPLSNGGNDDKNI
GGPIVTNLLTCGKPKLPPHALPPFYCCPPMSASEPIDFTFPDPSEPLRVRRPAHAVGAEYMAKYERAIALMKALPDSDPRS
FYQMANIHCAYCTGSYRQTAHRELNVQIHFSWFFFAFHRAYLYFFERIAAKLLGEPGFAVPFWSWDVPEGMRMPVEFANAS
              * * *
SPLYDPVRNPRHAPPKVVDLEFVRSSVDDKFTDEQQIQQNLRVMYKQMISNAALPSLFHGQPYRAGESDRPGAGTVELFPH
                                                                                                                                                                *
NTMHTWTGDLARPSVENMGVYYSAGRDPIFYPHHNNIDRLWEVWRDVGAARGYRGHVDFTDPDWLDSSFLFYDEEARLVRI
      * * *
TVRDVLDIDKLRYAYDGVGTPWLDAKPPATPNVNTKKGLLKSVRFPVSLDDVAVTAEVRRPRVLRSRREKEVQEEVLVIDG
IETDGADMVKFDVYVNAVEYEKVEPGGREMAGSFVCLMHPSMDGTGKGMGIQTSMRVALNELLEDLGADGDDSVTVTLVPR
NGKVSIGGLRIVYMME
```

H and Y amino acid residues indicated by an asterisk above are predicted to be key residues for metal ion (i.e., Cu²⁺) binding. In some embodiments, the PPO comprises one or more, or all of these indicated H and Y amino acid residues in their respective positions corresponding PPO (i.e., SEQ ID NO:1 to SEQ ID NO:3).

The amino acid sequence of BdTyDC2 (BdiBd21-3.2G0653800_TyDC2) is as follows:

```
(SEQ ID NO: 4)
MAPTSMCFDAINGAAAAQNGTAPVLATKPAAQALQCPNALNADDFRRQGHQVIDFIAEYYGGMADYPVHPSVTPGFLRNLL
PASAPSRAEPDAFSSALKDIRDHILPGMTHWQSPRHFAHFPASSSTVGALGEALTAGINVVPFTWAASPAATELEMVVVDW
LGKALHLPETLLFAGGGGGTLLGTSCEAILCALVAARDRKLAEIGGRRIGDLVVYCSDQTHFAFRKAARIAGILREHIREI
                                                                                                                ++++*
QTCHANMFALSATALEAAMQADVEAGLVPLFVCATVGTTQTTAVDPIGELCTVTAPHGVWVHVDAAYAGSALVCPEFRHVI
                                                                                                                          *++
NGVESVDSFSMNAHKWLLTNNDCCAMWVKKPSELIAALGTEQEYILKDSASEGHDIVDYKDWTMTLTRRFRALKMWLVLRC
  *++
YGIDGLREHIRSHVRMAEAFENLVRADERFEVVTDRQFALVCFRLRSPEKYGGEKTANELNRSLLEEVNAVTLGPYMSSAN
VGGMYMLRCAVGSTLTEDCHVTDGWKVVQDRATSILRKMEIIYSVLG
```

The amino acid sequence of BdTyDC3 (BdiBd21-3.2G0654700_TyDC3) is as follows:

```
(SEQ ID NO: 5)
MAPPSHFSNVAATVPVVVDKPQQCSNALDADDFRRQGHQVIDFIAEYYGGMADYPVHPSVTPGFLRNLLPASAPSRAEPDA
FSSALKDIRDHILPGMTHWQSPRHFAHFPASSSTVGALGEALTAGINVVPFTWAASPAATELEMVVVDWLGKALHLPETLL
FAGGGGGTLLGTSCEAILCALVAARDRKLAEIGGRRIGDLVVYCSDQTHFAFRKAARIAGILREHIREIQTCHANMFALSA
                                                                                        ++++*
TALEAAMQADVEAGLVPLFVCATVGTTQTTAVDPIGELCTVTAPHGVWVHVDAAYAGSALVCPEFRHVINGVESVDSFSMN
                                                                                                  *++
AHKWLLTNNDCCAMWVKKPSELIAALGTEQEYILKDSASEGHDIVDYKDWTMTLTRRFRALKMWLVLRCYGIDGLREHIRS
  *++
HVRMAEAFENLVRADERFEVVTDRQFALVCFRLRSPEKYGGEKTANELNRSLLEEVNAVTLGPYMSSANVGGMYMLRCAVG
STLTEDCHVTDGWKVVQDRATSILRKMEIIYSVLG
```

Tyrosine decarboxylases (TyDCs) from other plant species have been characterized. Two TyDCs identified herein from Brachypodium have similar enzymatic activities as previously characterized TyDCs. H amino acid residues indicated by an asterisk above, and the amino acid residues (indicated by a plus sign above) adjacent or near these H amino acid residues are predicted to be important for metal ion binding, or the enzymatic activity. In some embodiments, the TyDC comprises one or more, or all of these indicated H and/or other amino acid residues in their respective positions corresponding TyDC (i.e., SEQ ID NO:4 and SEQ ID NO:5).

All of the references cited herein are each incorporated by reference. References cited herein include:
1. Olguín HJ, Guzmán DC, García EH, and Barragan G. The role of dopamine and its dysfunction as a consequence of oxidative stress. Oxidative Medicine and Cellular Longevity. 2016, 2016:9730467
2. Liu Q, Gao T, Liu W, Liu Y, Zhao Y, Liu Y, Li W, Ding K, Ma F, Li C. Functions of dopamine in plants: a review. Plant Signal Behav. 2020, 15:1827782
3. Galanie S, Thodey K, Trenchard I J, Filsinger Interrante M, Smolke C D. Complete biosynthesis of opioids in yeast. Science. 2015, 349:1095-100
4. Ryu J H, Messersmith P B, Lee H. Polydopamine surface chemistry: A decade of discovery. ACS Appl Mater Interfaces. 2018, 10:7523-7540
5. Li F, Yu Y, Wang Q, Yuan J, Wang P, Fan X. Polymerization of dopamine catalyzed by laccase: Comparison of enzymatic and conventional methods. Enzyme and Microbial Technology. 2018, 19:58-64
6. de Nobili M, Bravo C, Chen Y. The spontaneous secondary synthesis of soil organic matter components: A critical examination of the soil continuum model theory. Appl Soil Ecol. 2020, 154:103655

In some embodiments, when the promoter is a tissue-specific promoter. Examples of tissue-specific promoters under developmental control include promoters that initiate transcription only (or primarily only) in certain tissues, such as vegetative tissues, cell walls, including e.g., roots or leaves. A variety of promoters specifically active in vegetative tissues, such as leaves, stems, roots and tubers are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarn promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are kn1-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

In some embodiments, the promoter is substantially identical to the native promoter of a promoter that drives expression of a gene involved in secondary wall deposition. Examples of such promoters are promoters from IRX1, IRX3, IRX5, IRX8, IRX9, IRX14, IRX7, IRX10, GAUT13, or GAUT14 genes. Specific expression in fiber cells can be accomplished by using a promoter such as the NST1 promoter and specific expression in vessels can be accomplished by using a promoter such as VND6 or VND7. (See, e.g., PCT/US2012/023182 for illustrative promoter sequences). In some embodiments, the promoter is a secondary cell wall-specific promoter or a fiber cell-specific promoter. In some embodiments, the promoter is from a gene that is co-expressed in the lignin biosynthesis pathway (phenylpropanoid pathway). In some embodiments, the promoter is a C4H, C3H, HCT, CCR1, CAD4, CAD5, F5H, PAL1, PAL2, 4CL1, or CCoAMT promoter. In some embodiments, the tissue-specific secondary wall promoter is an IRX1, IRX3, IRX5, IRX8, IRX9, IRX14, IRX7, IRX10, GAUT13, GAUT14, or CESA4 promoter. Suitable tissue-specific secondary wall promoters, and other transcription factors, promoters, regulatory systems, and the like, suitable for this present invention are taught in U.S. Patent Application Pub. Nos. 2014/0298539, 2015/0051376, and 2016/0017355.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Plant Dopamine Biosynthetic Pathway

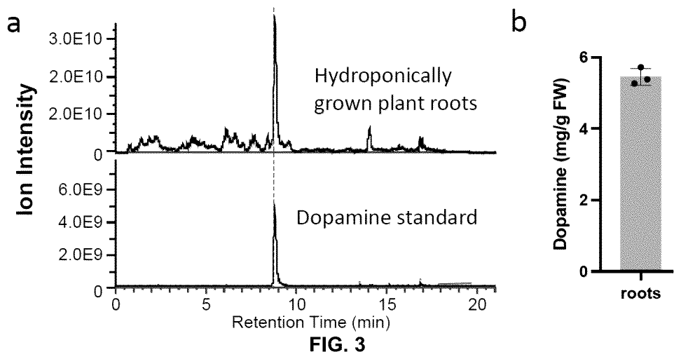

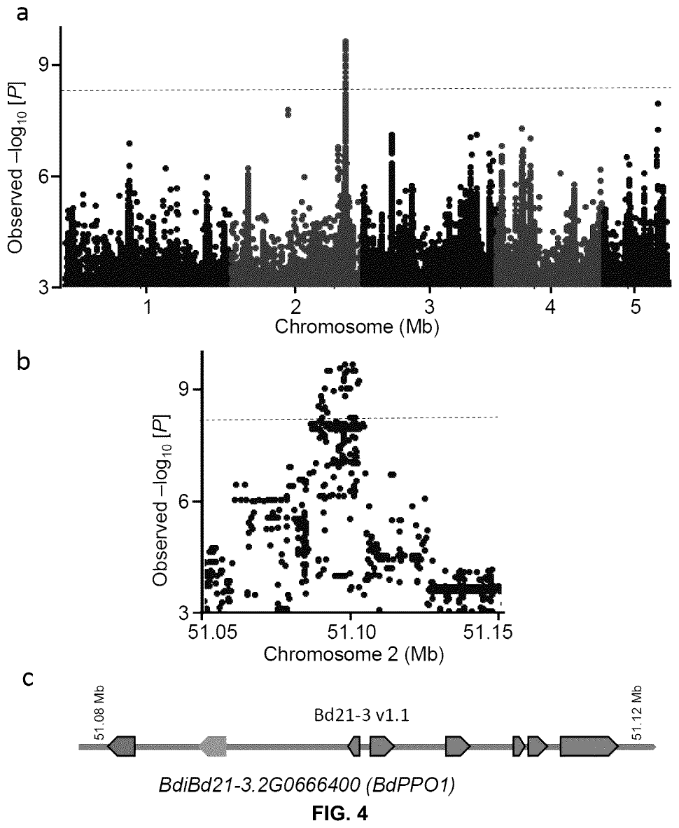

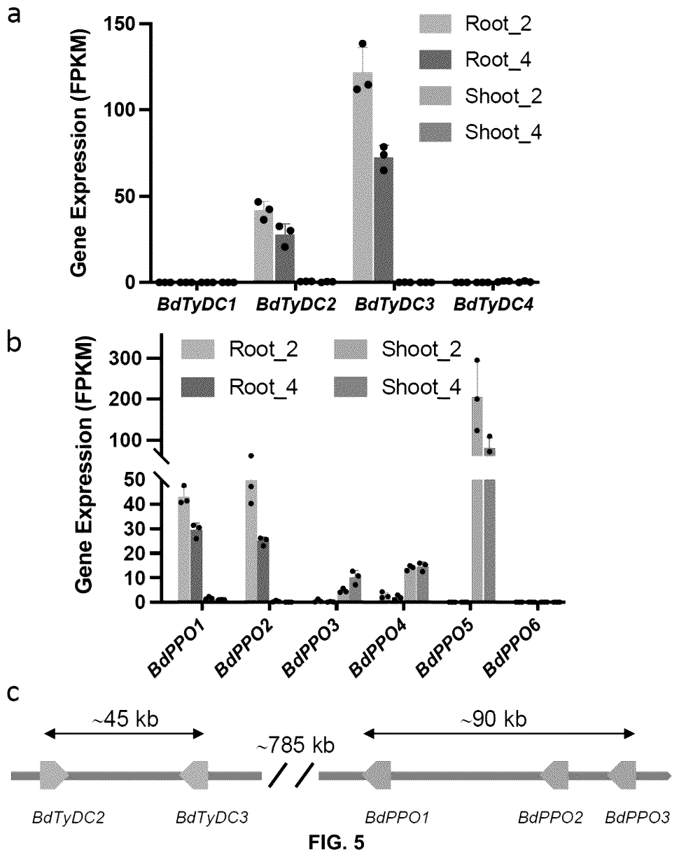

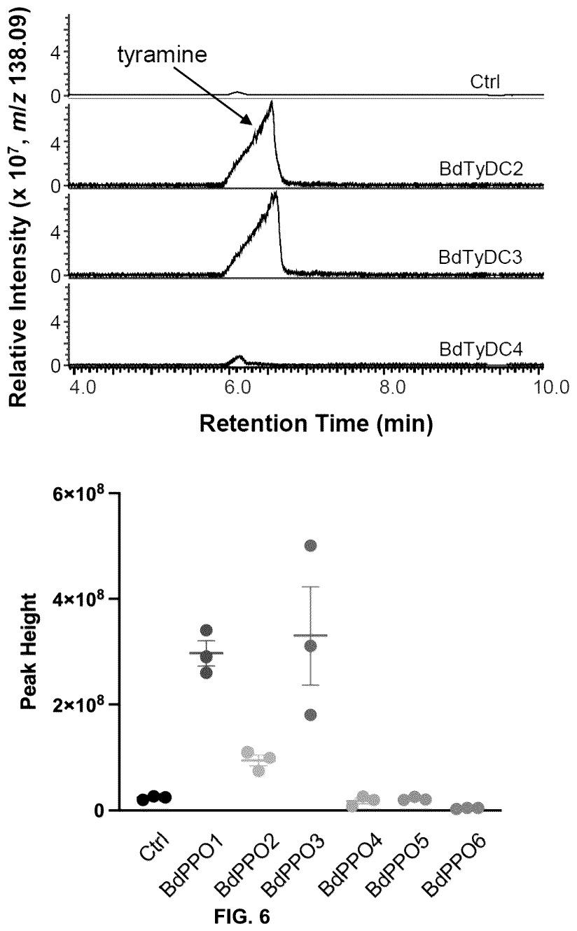

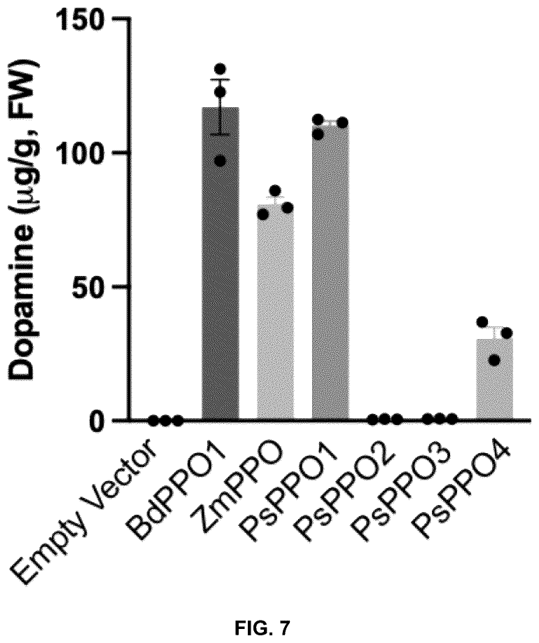

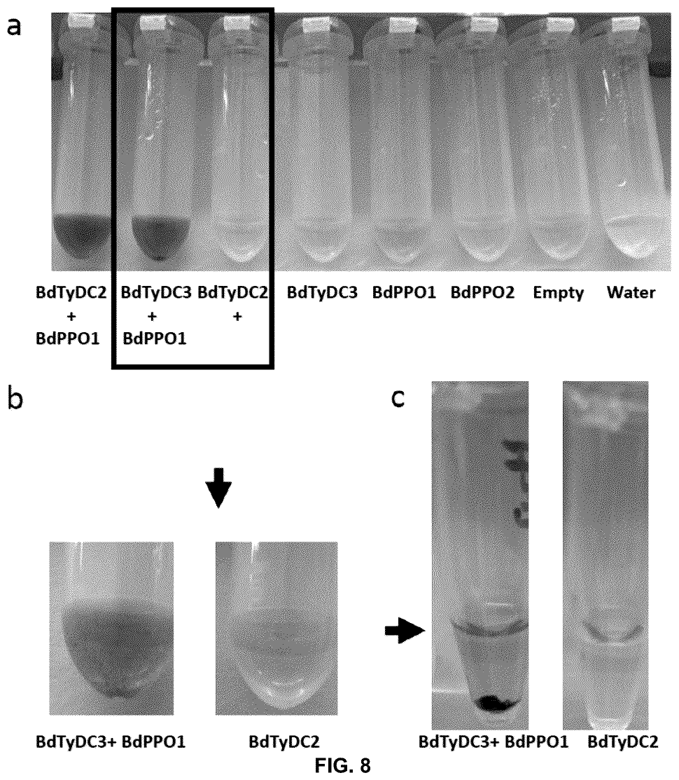

During our study, we found that the grass model plant, Brachypodium distachyon, could produce exceedingly high levels of dopamine (>5 mg/g Fresh Weight; FIG. 3) under certain growth conditions. By leveraging multi-omics data analysis in conjunction with metabolite-based genome-wide association studies, we successfully identified candidate enzymes in Brachypodium for dopamine biosynthesis, including two tyrosine decarboxylases (BdTyDC2 & 3) and three polyphenol oxidases (BdPPO1, 2, & 3) (FIG. 4 and FIG. 5). Interestingly, these enzyme-encoding genes form a cluster on chromosome 2 in Brachypodium distachyon (FIG. 5). We also found that the dopamine biosynthetic gene cluster is conserved in plants capable of synthesizing dopamine (FIG. 5). Transcriptomic data analysis revealed that these genes except BdPPO3 are predominantly expressed in Brachypodium roots (FIG. 5). Subsequent in vivo enzymatic assays confirmed that a combination of BdTyDC2 or BdTyDC3 with BdPPO1, BdPPO2, or BdPPO3 leads to dopamine production (FIG. 6), confirming that these enzymes are involved in plant dopamine biosynthesis. We also found that these enzyme homologs in other plant species have similar enzymatic activities (FIG. 7).

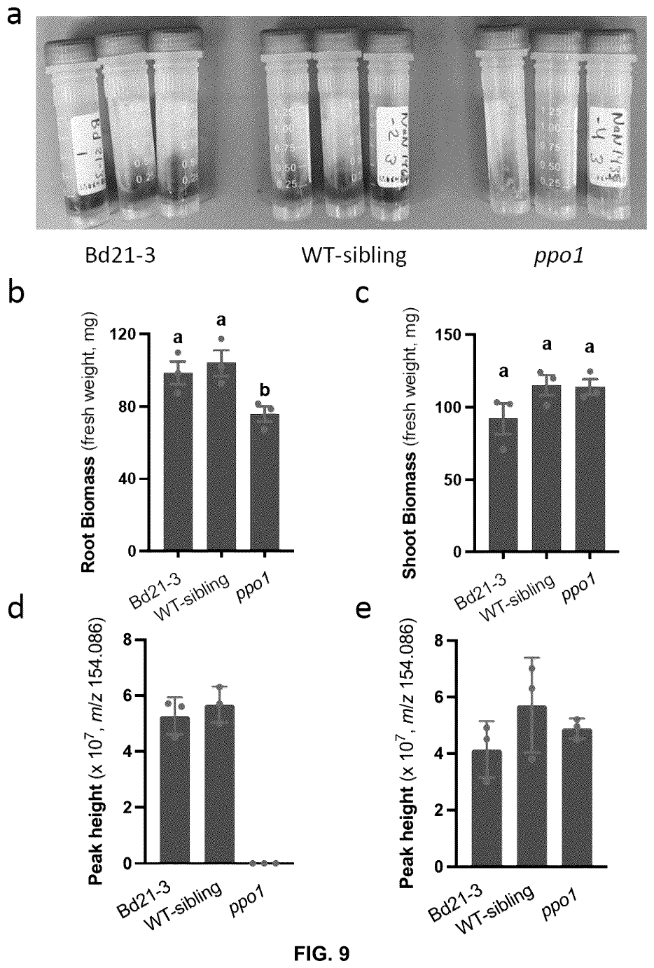

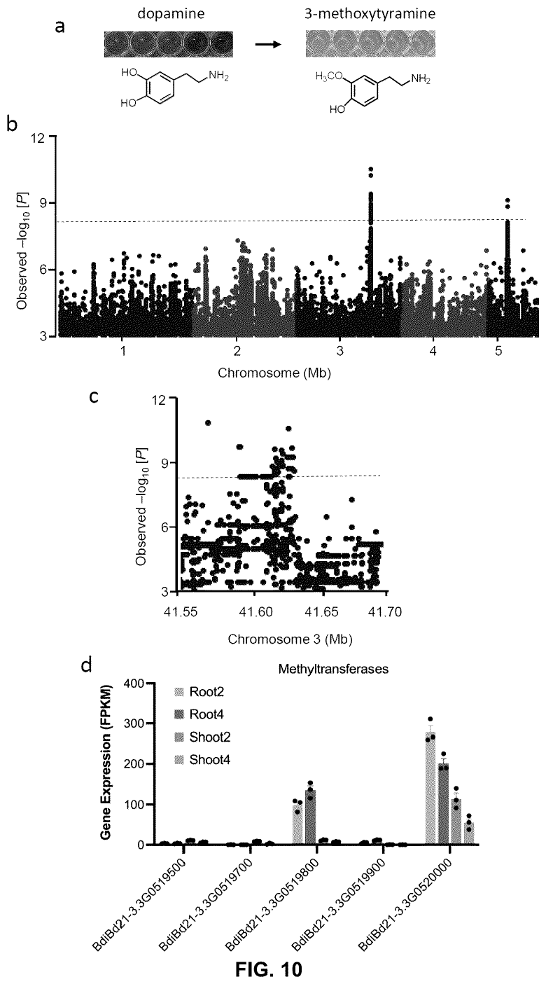

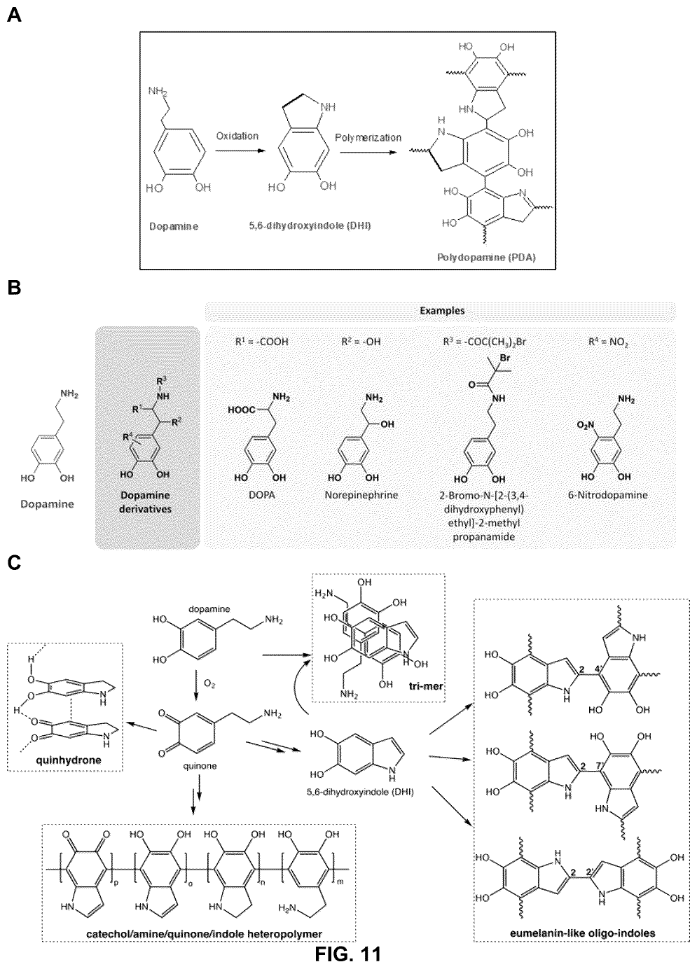

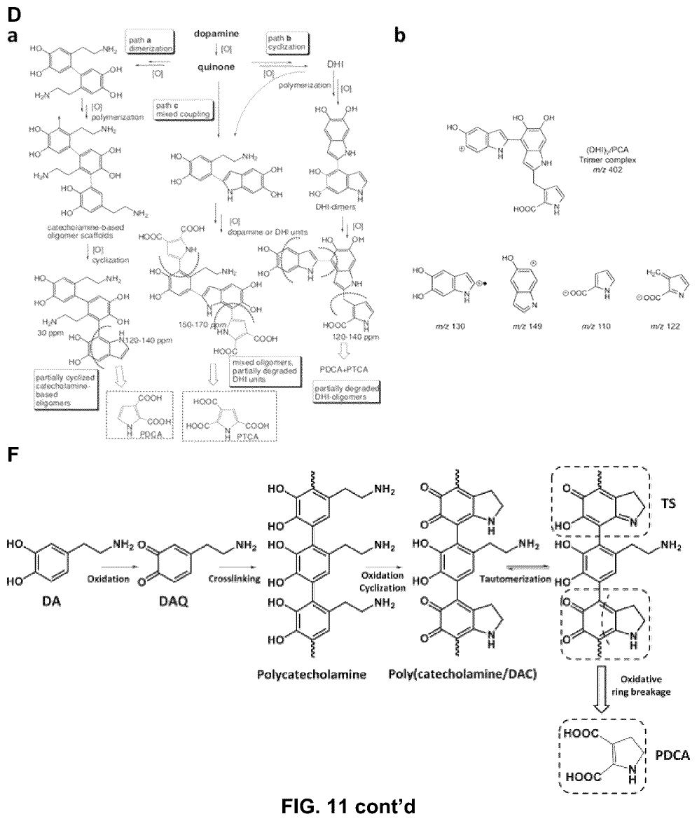

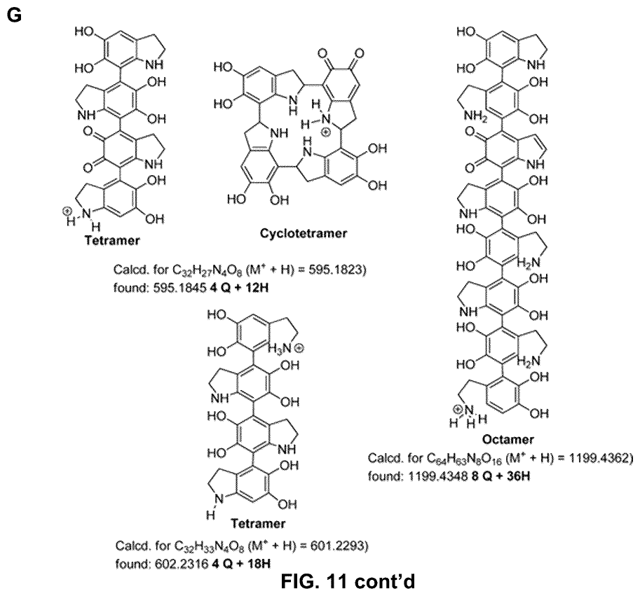

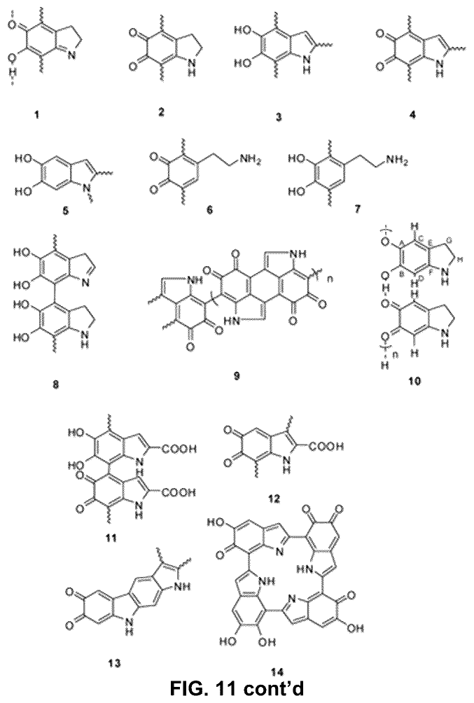

We identified a Brachypodium Bdppo1 mutant line, which carries a premature stop codon. Metabolic profiling revealed that the Bdppo1 mutant roots are deficient in producing dopamine, while the shoots produce dopamine at the wildtype level, suggesting that BdPPO1 is only responsible for root dopamine biosynthesis (FIG. 9). Consistently, we observed that the mutation had an impact only on the growth of roots and not on shoots. Interestingly, we found that the Bdppo1 mutant plants did not produce a brown color after damage (FIG. 9), indicating a lack of production of dopamine polymers. Additionally, we also identified two candidate methyltransferase-encoding genes, which convert dopamine to 3-methoxytyramine (FIG. 10). These candidate methyltransferases are likely involved in a process to avoid the formation of dopamine polymers (FIG. 10).

The discovery of dopamine pathway enzymes allows us to bioengineer the pathway for the following purposes. Notably, chemical inducible and/or senescence-related promoters will be used to drive the expression of the pathway in both microbes and plants. The methyltransferases identified in this study, along with their homologs, can be used as a potential solution for safeguarding cells against the overaccumulation of dopamine caused by promoter leaking.

### Materials and Methods

### Plant Materials

Seeds of 99 Brachypodium distachyon accessions were obtained from the Vogel laboratory at Joint Genome Institute (JGI). These accessions were grown hydroponically in a Fabrication System for 4 weeks before harvesting. Brachypodium root and shoot samples were frozen in liquid nitrogen, ground to fine powders, and stored at −80° C. for further analyses.

### Metabolomics Analysis and Data Processing

Frozen samples were extracted with 500 mL of 70% (v/v) methanol acidified with 0.1% (v/v) formic acid and analyzed using a Q Exactive Hybrid Quadrupole-Orbitrap Mass Spectrometer after separation with normal phase (hydrophilic interaction liquid chromatography, HILIC) liquid chromatography (Brisson et al., 2022). Raw MS data were processed and analyzed using MZmine2. The dopamine pathway compounds, such as tyramine and dopamine, were identified based on known masses and chemical standards.

Metabolite-Based Genome-Wide Association Studies (mGWASs)

mGWASs were conducted with levels of tyramine and dopamine in roots as well as the ratio of dopamine to tyramine as mapping traits using the SNP (single nucleotide polymorphism) data of the 99 Brachypodium accessions provided by the Vogel laboratory. mGWASs were performed using the Unified Mixed Linear Model (MLM) in TASSEL 5.0 as previously described (Ding et al., 2017). SNPs with less than 20% missing genotype data and minor allele frequencies>5% were employed in the association analysis. Manhattan plots were constructed in the R package qqman (v0.1.4) (http://cran.rproject.org/web/packages/qqman).

### In Vivo Enzymatic Assay

Candidate gene function was examined using transient expression assays in N. benthamiana as previously described (Ding et al., 2019 & 2020). Briefly, full-length open reading frames of Brachypodium TyDC and BdPPO family genes were amplified from root cDNA of the Brachypodium line Bd21-3 and cloned onto the plant expressing vector, pLIFE33, which were then transformed into Agrobacterium tumefaciens strain GV3101. BdPPO1 homologs were synthesized with codon-optimized sequences. Resulting Agrobacterium cells were grown at 28° C. for 24 h in Luria broth media supplemented with 50 mg L⁻¹ of kanamycin, 30 mg L⁻¹ of gentamicin, and 50 mg L⁻¹ of rifampicin. Cells were collected and resuspended to a final optical density 600 of 1.0 in 10 mM MES buffer with 10 mM MgCl₂. Equal volumes of different cultures were combined and infiltrated into the leaves of 5-week-old N. benthamiana plant leaves using a needleless syringe. Three days post-transfection, inoculated leaves were collected for metabolite analysis.

RNA Extraction and cDNA Library Construction

Total RNA was isolated using the NucleoSpin RNA Plant Kit (Takara Bio) according to the manufacturer's protocol. RNA quality was assessed on the basis of RNA integrity number using an Agilent Bioanalyzer. Total RNA was used for the construction of a 5′ rapid amplification of cDNA ends (RACE) cDNA library with the SMARTer RACE 5′/3′ Kit (Clontech) in accordance with the manufacturer's protocol. Full-length open reading frames were then amplified using gene-specific oligonucleotides.

### RNA-Seq Analyses

RNA-seq analysis was performed as previously described (Ding et al., 2019 & 2020). Briefly, libraries were constructed using an Illumina TruSeq Stranded RNA LT Kit following manufacturer protocols. For RNA-seq data analysis, raw fastq reads were trimmed for adaptors and preprocessed to remove low quality reads using Trimmomatic v.0.32. Qualified reads were then aligned to the Brachypodium distachyon Bd21-3 v1.1 reference genome using Bowtie2 v.2.2.3.0 and TopHat v.2.0.13. Gene expression values were computed using Cufflinks v.2.2.1.

### REFERENCES

Brisson, V. L., Richardy, J., Kosina, S. M., Northen, T. R., Vogel, J. P., Gaudin, A. C. M. Phosphate Availability Modulates Root Exudate Composition and Rhizosphere Microbial Community in a Teosinte and a Modern Maize Cultivar. Phytobiomes J. 6, 83-94 (2022).
Ding, Y., Huffaker, A., K. llner, T. G., Weckwerth, P., Robert, C A. M., Spencer, J. L., Lipka, A. E., Schmelz, E. A. Selinene Volatiles Are Essential Precursors for Maize Defense Promoting Fungal Pathogen Resistance. Plant Physiol. 175, 1455-1468 (2017).
Ding, Y., et al. Multiple genes recruited from hormone pathways partition maize diterpenoid defences. Nat. Plants. 5, 1043-1056 (2019).
Ding, Y., et al. Genetic elucidation of interconnected antibiotic pathways mediating maize innate immunity. Nat. Plants. 6, 1375-1388 (2020).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified plant or plant cell capable of producing dopamine or dopamine-derived compound, comprising one or more heterologous tyrosine decarboxylases (TyDC) or one or more heterologous polyphenol oxidases (PPO).

2. A method for constructing a genetically modified plant or plant cell of the present invention, comprising: (a) introducing one or more nucleic acids or polynucleotides comprising the open reading frame(s) (ORF) encoding one or more heterologous tyrosine decarboxylases (TyDC) or one or more heterologous polyphenol oxidases (PPO), wherein each ORF is operatively linked to a promoter capable of expressing the TyDC(s) and/or PPO(s) into the plant or plant cell; and (b) the one or more nucleic acids or polynucleotides stably reside in the plant or plant cell.

3. A method for producing dopamine or dopamine-derived compound, comprising: (a) providing a genetically modified plant or plant cell of the present invention; (b) culturing or growing the genetically modified plant or plant cell thereby the genetically modified plant or plant cell produces dopamine or dopamine-derived compound; and, (c) optionally separating or purifying the dopamine or dopamine-derived compound from the rest of the genetically modified plant or plant cell.
